(19) United States
(12) Patent Application Publication
(10) Pub. No.: US 2022/0089740 A1
(43) Pub. Date: Mar. 24, 2022

(54) TIM-3 ANTIBODIES AND COMBINATIONS WITH OTHER CHECKPOINT INHIBITORS FOR THE TREATMENT OF CANCER

(71) Applicant: Eli Lilly and Company, Indianapolis, IN (US)

(72) Inventors: Mythili KONERU, Magnolia, TX (US); Maria de las Nieves VELEZ DE MENDIZABAL CASTILLO, Zionsville, IN (US)

(21) Appl. No.: 17/420,189

(22) PCT Filed: Jan. 3, 2020
(86) PCT No.: PCT/US2020/012118

§ 371 Date: Jul. 1, 2021

Related U.S. Application Data
(60) Provisional application No. 62/791,077, filed on Jan. 11, 2019.

(51) Int. Cl. C07K 16/28; C07K 16/30; A61P 35/00

(52) U.S. Cl. CPC C07K 16/2827; A61K 2039/505; A61P 35/00; C07K 16/30

(57) ABSTRACT

The present invention relates to dosing regimens for antibodies that bind human T-cell immunoglobulin- and mucin-domain-containing protein-3 (TIM-3), and may be useful for treating tumors in combination with anti-human PD-L1 antibodies, anti-human PD-1 antibodies, chemotherapy, and ionizing radiation as well as the use of said antibodies for the treatment of solid tumors that are mismatch repair deficient or exhibit a high degree of microsatellite instability.

The present invention relates to the use of anti-human human T-cell immunoglobulin- and mucin-domain-containing protein-3 (TIM-3) antibodies for the treatment of cancer. More particularly, the present invention relates to dosing regimens for the use of anti-human TIM-3 antibodies for the treatment of cancer alone or in combination with other checkpoint inhibitors, such as an anti-human programmed cell death 1 ligand 1 (PD-L1) antibody or an anti-human programmed cell death protein 1 (PD-1) antibody.

Tumor cells escape detection and elimination by the immune system through multiple mechanisms some of which include the manipulation of immune checkpoint pathways. Immune checkpoint pathways are used in self-tolerance maintenance and in the regulation of T cell activation, but cancer cells can manipulate these pathways to prolong tumor survival. The PD-1/PD-L1 pathway is one such immune checkpoint. In addition to the PD-1/PD-L1 pathway, T cells recognizing tumor antigens can also express other checkpoint receptors, such as TIM-3. In particular, T cells expressing TIM-3 can exhibit an exhausted phenotype characterized by an impairment in cytotoxic functions, effector cytokine production, and proliferation. In this regard, it has been shown that anti-TIM-3 antibodies can restore anti-tumor immunity in some murine cancer models. Morever, it has also been shown that some patients who develop adaptive resistance to anti-PD-1 treatment display an upregulation of TIM-3 on their T cells.

Antibodies directed to human TIM-3 are known and have been described in WO2018/039020. An anti-human TIM-3 antibody described in WO2018/039020 is currently being tested in human clinical trials as a single agent and in combination with an anti-human PD-L1 antibody previously described in WO2017/034916 (NCT03099109). However, no antibody targeting TIM-3 has received regulatory approval for therapeutic use in humans alone or in combination with an anti-human PD-L1 or anti-human PD-1 antibody.

There remains a need for dosing regimens for anti-human TIM-3 antibodies. There also remains a need for dosing regimens for anti-human TIM-3 antibodies in combination with anti-PD-L1 antibodies or anti-human PD-1 antibodies, for the treatment of cancer. In particular, there remains a need for dosing regimens for anti-human TIM-3 antibodies that optimize clinical benefit over clinical risk, such as regimens that produce nearly complete cell surface occupancy of TIM-3 or soluble TIM-3 target engagement during both the loading and maintenance phases for the patient using the regimen. There also remains a need to minimize the risk of non-linear pharmacokinetics in the tumor environment of patients using the regimen.

Accordingly, some embodiments of the present invention include dosing regimens for anti-human TIM-3 antibodies for the treatment of cancer. Further, some embodiments of the present invention include dosing regimens where the cancer is a solid tumor. Embodiments of the present invention also include dosing regimens for the combination of anti-human TIM-3 antibodies and anti-human PD-L1 or anti-human PD-1 antibodies for the treatment of cancer. Further, embodiments of the present invention also include dosing regimens for the combination of anti-human TIM-3 antibodies and anti-human PD-L1 or anti-human PD-1 antibodies for the treatment of cancer, wherein the cancer is a solid tumor.

Further, in some embodiments of the present invention, the combination of anti-human TIM-3 antibodies and anti-human PD-L1 or anti-human PD-1 antibodies as part of a treatment regimen for solid tumors are for patients whose tumors are MSI-H or MMR deficent. Embodiments of the present invention also include anti-human TIM-3 antibodies as a treatment regimen for solid tumors in patients whose tumors are MSI-H or MMR deficient and have previously been treated with an anti-human PD-L1 antibody or an anti-human PD-1 antibody. Embodiments of the present invention also include the combination of anti-human TIM-3 antibodies and anti-human PD-L1 or anti-human PD-1 antibodies as a treatment regimen for advanced solid tumors in patients whose tumors are MSI-H or MMR deficient and have previously been treated with an anti-human PD-L1 antibody or an anti-human PD-1 antibody. Embodiments of the invention also include anti-human TIM-3 antibodies as a treatment regimen for advanced solid tumors in patients whose tumors are MSI-H or MMR deficient and have not been previously treated with an anti-human PD-L1 antibody or an anti-human PD-1 antibody. Some embodiments of the present invention also include the combination of anti-human TIM-3 antibodies and anti-human PD-L1 or anti-human PD-1 antibodies as a treatment regimen for solid tumors in patients whose tumors are MSI-H or MMR deficient and have not been previously treated with an anti-human PD-L1 antibody or an anti-human PD-1 antibody.

Some embodiments of the present invention also include dosing regimens for the combination of anti-human TIM-3 antibodies and anti-human PD-L1 or anti-human PD-1 antibodies for the treatment of cancer, wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, liver cancer, gallbladder cancer, cervical cancer, duodenal cancer, bone cancer, neuroendocrine cancer, intestinal cancer.

In some embodiments of the present invention, the anti-human TIM-3 antibody binds the extracellular domain of human TIM-3 (SEQ ID NO: 1). Further, in some embodiments of the present invention, the anti-human TIM-3 antibody comprises a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7. In some embodiments, the anti-human TIM-3 antibody comprises a light chain variable region having the amino acid sequence of SEQ ID NO: 8 and a heavy chain variable region having the amino acid sequence of SEQ ID NO: 9. In some embodiments, the anti-human TIM-3 antibody comprises a light chain having the amino acid sequence of SEQ ID NO: 10 and a heavy chain having the amino acid sequence of SEQ ID NO: 11.

In some embodiments, the anti-human TIM-3 antibody blocks the binding of human TIM-3 to human phosphatidylserine, but does not block binding of human TIM-3 to human CEACAM1. In some embodiments, the anti-human TIM-3 antibody blocks the binding of human TIM-3 to human phosphatidylserine, but does not block the binding of human TIM-3 to human CEACAM1, and also blocks the binding of human TIM-3 to human galectin-9. Anti-human TIM-3 antibodies that block the binding of human TIM-3 to human phosphatidylserine, but do not block the binding of human TIM-3 to human CEACAM1, and also block binding of human TIM-3 to human galectin-9 have been previously described in WO2018/039020 and include antibodies that comprise a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7.

Non-limiting examples of anti-human PD-L1 antibodies for use in the combinations of the present invention include atezolizumab, durvalumab, avelumab, BMS-936559, and preferably those described in WO2017/034916. In some examples, the anti-human PD-L1 antibody comprises a light chain having the amino acid sequence of SEQ ID NO: 12 and a heavy chain having the amino acid sequence of SEQ ID NO: 13

Non-limiting examples of anti-human PD-1 antibodies for use in the combinations of the present invention include those as described in WO2017025016, PDR001 (described in US20150210769; CAS registry number 1859072-53-9), MEDI0680, REGN2810, BGB-A317, nivolumab (CAS registry number 946414-94-4), pembrolizumab (CAS registry number 1374853-91-4), TSR-042, and those described in WO18085468.

Further, some embodiments of the present invention include the combination of chemotherapeutic agents. Non-limiting examples of useful chemotherapeutic agents for use herein include 5-fluorouracil, hydroxyurea, gemcitabine, methotrexate, doxorubicin, etoposide, carboplatin, cisplatin, cyclophosphamide, melphalan, dacarbazine, taxol, camptothecin, FOLFIRI, FOLFOX, docetaxel, daunorubicin, paclitaxel, oxaliplatin, and combinations thereof.

The present invention is derived from two Phase 1a/b clinical trials: a phase 1a/1b study of an anti-human TIM-3 antibody, administered alone or in combination with an anti-human PD-L1 antibody, in advanced relapsed/refractory solid tumors (NCT03099109) and a phase 1a/1b study of a novel anti-human PD-L1 checkpoint antibody administered alone or in combination with other agents in advanced refractory solid tumors (NCT02791334).

The present invention provides a dosing regimen for the use of an anti-human TIM-3 antibody for the treatment of cancer, wherein the anti-human TIM-3 antibody is administered at a dose of 1 mg to 1800 mg, 30 mg to 1800 mg, 30 mg to 1800 mg once every three weeks, 30 mg to 1800 mg once every two weeks, 30 mg to 1200 mg once every two weeks, 30 mg to 900 mg once every three weeks, 30 mg to 600 mg once every two weeks, 1 mg once every two weeks, 10 mg once every two weeks, 30 mg once every two weeks, 70 mg once every two weeks, 200 mg once every two weeks, 600 mg once every two weeks, 900 mg once every three weeks about 1000 mg once every three weeks, 1200 mg once every three weeks, or 1800 mg once every three weeks, and wherein the anti-human TIM-3 antibody comprises:
A.) a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7,
B.) a light chain variable region having the amino acid sequence of SEQ ID NO: 8 and a heavy chain variable region having the amino acid sequence of SEQ ID NO: 9, or
C.) a light chain having the amino acid sequence of SEQ ID NO: 10 and a heavy chain having the amino acid sequence of SEQ ID NO: 11.

The present invention provides a method of treating cancer comprising administering an anti-human TIM-3 antibody to a human patient in need thereof with a dose in the range of 1 mg to 1800 mg, wherein the anti-human TIM-3 antibody comprises a light chain having the amino acid sequence of SEQ ID NO: 10 and a heavy chain having the amino acid sequence of SEQ ID NO: 11.

In some embodiments, the dose is administered once every two weeks. Further, in some embodiments, the dose is administered once every two weeks and the dose is in the range of 30 mg to 1200 mg, the dose is 30 mg, the dose is 70 mg, the dose is 200 mg, or the dose is 600 mg.

In some embodiments, the dose is administered once every three weeks. Further, in some embodiments, the dose is administered once every three weeks and the dose is in the range of 30 mg to 1800 mg, the dose is 900 mg, the dose is 1000 mg, the dose is 1200 mg, or the dose is 1800 mg.

In some embodiments of the present invention, the anti-TIM-3 antibody is administered with a loading dose followed by a maintenance dose, wherein the loading dose is administered once every two to three weeks for one to three cycles, the maintenance dose is administered once every two to three weeks following completion of the loading dose, the loading dose and the maintenance doses are in the range of 1 mg to 1800 mg, the maintenance dose is a lower dosage amount than the loading dose, and wherein the anti-human TIM-3 antibody comprises:
A.) a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7,
B.) a light chain variable region having the amino acid sequence of SEQ ID NO: 8 and a heavy chain variable region having the amino acid sequence of SEQ ID NO: 9, or
C.) a light chain having the amino acid sequence of SEQ ID NO: 10 and a heavy chain having the amino acid sequence of SEQ ID NO: 11.

Further, in some embodiments, the maintenance dose is half the dosage amount of the loading dose. In some embodiments, the loading dose is 1200 mg administered once every two weeks for two cycles, and the maintenance dose is 600 mg administered once every two weeks. In some embodiments, the loading dose is 1800 mg administered once every three weeks for three cycles, and the maintenance dose is 900 mg administered once every three weeks. Additionally, in some embodiments, the maintenance dose is administered up to two years.

The present disclosure provides a method of treating cancer comprising administering to a human patient whose cancer comprises a solid tumor that is mismatch repair deficient or exhibits a high degree of microsatellite instability, an effective amount of an anti-human TIM-3 (SEQ ID NO: 1) antibody.

The present disclosure provides a method of treating cancer comprising administering to a human patient whose cancer comprises a solid tumor that is mismatch repair deficient or exhibits a high degree of microsatellite instability, an effective amount of an anti-human TIM-3 (SEQ ID NO: 1) antibody; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody.

The present disclosure provides a method of treating cancer comprising administering to a human patient whose cancer comprises a solid tumor that is mismatch repair deficient or exhibits a high degree of microsatellite instability, an effective amount of an anti-human TIM-3 (SEQ ID NO: 1) antibody; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7.

The present disclosure provides a method of treating cancer comprising administering to a human patient whose cancer comprises a solid tumor that is mismatch repair deficient or exhibits a high degree of microsatellite instability, an effective amount of an anti-human TIM-3 (SEQ ID NO: 1) antibody; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a light chain variable region having the amino acid sequence of SEQ ID NO: 8 and a heavy chain variable region having the amino acid sequence of SEQ ID NO: 9.

The present disclosure provides a method of treating cancer comprising administering to a human patient whose cancer comprises a solid tumor that is mismatch repair deficient or exhibits a high degree of microsatellite instability, an effective amount of an anti-human TIM-3 (SEQ ID NO: 1) antibody; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a light chain having the amino acid sequence of SEQ ID NO: 10 and a heavy chain having the amino acid sequence of SEQ ID NO: 11.

The present disclosure provides a method of treating cancer comprising administering an anti-human TIM-3 antibody to a human patient in need thereof, wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody, and wherein the anti-human PD-L1 antibody is administered at a dose of about 35 mg to about 1800 mg, about 70 mg to about 1400 mg, about 35 mg once every two weeks, about 70 mg once every three weeks, about 200 mg once every two weeks, 700 mg once every two weeks, about 1000 mg to about 100 mg, about 1000 mg once every three weeks, about 1400 mg once every three weeks, or about 1800 mg once every three weeks.

The present disclosure provides a method of treating cancer comprising administering an anti-human TIM-3 antibody to a human patient in need thereof, wherein the anti-human TIM-3 antibody is administered in combination with an effective amount of an anti-human PD-L1 (SEQ ID NO: 14) antibody, wherein the anti-human PD-L1 antibody is administered at a dose of 35 mg to 1800 mg, 70 mg to 1400 mg, 35 mg once every two weeks, 70 mg once every three weeks, 200 mg once every two weeks, 700 mg once every two weeks, 1000 mg to 100 mg, 1000 mg once every three weeks, 1400 mg once every three weeks, or 1800 mg once every three weeks, and wherein the anti-human PD-L1 antibody comprises a light chain having the amino acid sequence of SEQ ID NO: 12 and a heavy chain having the amino acid sequence of SEQ ID NO: 13.

The present disclosure provides a method of treating cancer comprising administering an anti-human TIM-3 antibody to a human patient in need thereof, wherein the anti-human TIM-3 antibody is administered in combination with an effective amount of an anti-human PD-1 (SEQ ID NO: 15) antibody, wherein the anti-human PD-1 antibody is administered at 200 mg once every 3 weeks, 240 mg administered once every 2 weeks, or 480 mg once every 4 weeks.

In some examples, the human patient receiving treatment with the anti-human TIM-3 antibody alone or in combination with an anti-human PD-L1 antibody or an anti-human PD-1 antibody was previously treated with an anti-human PD-L1 antibody or an anti-human PD-1 antibody. In some examples, the human patient receiving treatment with the anti-human TIM-3 antibody alone or in combination with an anti-human PD-L1 antibody or an anti-human PD-1 antibody was not previously treated with an anti-human PD-L1 antibody or an anti-human PD-1 antibody. In some examples, the human patient receiving treatment with the anti-human TIM-3 antibody alone or in combination with an anti-human PD-L1 antibody or an anti-human PD-1 antibody has a solid tumor that is PD-L1 high. In some examples, the human patient receiving treatment with the anti-human TIM-3 antibody alone or in combination with an anti-human PD-L1 antibody or an anti-human PD-1 antibody has a solid tumor that is PD-L1 low. In some examples, the human patient receiving treatment with the anti-human TIM-3 antibody alone or in combination with an anti-human PD-L1 antibody or an anti-human PD-1 antibody has a solid tumor that has a high degree of microsatellite instability. In some examples, the human patient receiving treatment with the anti-human TIM-3 antibody alone or in combination with an anti-human PD-L1 antibody or an anti-human PD-1 antibody has a solid tumor that is mismatch repair deficient.

In some examples, the human patient receiving treatment with the anti-human TIM-3 antibody alone or in combination with an anti-human PD-L1 antibody or an anti-human PD-1 antibody has a cancer that is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, liver cancer, gallbladder cancer, cervical cancer, duodenal cancer, bone cancer, neuroendocrine cancer, intestinal cancer. In some examples, the human patient receiving treatment with the anti-human TIM-3 antibody alone or in combination with an anti-human PD-L1 antibody or an anti-human PD-1 antibody has a cancer that is melanoma or non-small cell lung cancer.

In some examples, the human patient receives ionizing radiation in simultaneous, separate, or sequential combination with their treatment with the anti-human TIM-3 antibody alone or in combination with an anti-human PD-L1 antibody or an anti-human PD-1 antibody. In some examples, the human patient receives one or more chemotherapeutic agents in simultaneous, separate, or sequential combination with their treatment with the anti-human TIM-3 antibody alone or in combination with an anti-human PD-L1 antibody or an anti-human PD-1 antibody.

In some examples, the anti-human TIM-3 antibody for use herein blocks binding of human TIM-3 to human phosphatidylserine, but does not block binding of human TIM-3 to human CEACAM1. In some examples, the anti-human TIM-3 antibody for use of herein blocks binding of human TIM-3 to human phosphatidylserine, but does not block binding of human TIM-3 to human CEACAM1, but also blocks binding of human TIM-3 to human galectin-9.

The present invention provides an anti-human TIM-3 (SEQ ID NO: 1) antibody for use in the treatment of a human patient whose cancer comprises a solid tumor. The present invention provides an anti-human TIM-3 antibody for use in the treatment of cancer.

The present invention provides an anti-human TIM-3 (SEQ ID NO: 1) antibody for use in the treatment of cancer, wherein the anti-human TIM-3 antibody is administered at a dose in the range of 1 mg to 1800 mg, and the anti-human TIM-3 antibody comprises:
A.) a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7,
B.) a light chain variable region having the amino acid sequence of SEQ ID NO: 8 and a heavy chain variable region having the amino acid sequence of SEQ ID NO: 9, or
C.) a light chain having the amino acid sequence of SEQ ID NO: 10 and a heavy chain having the amino acid sequence of SEQ ID NO: 11.

In some embodiments of the present invention, the dose is administered once every two weeks. Further, in some embodiments, the dose is administered once every two weeks and the dose is in the range of 30 mg to 1200 mg, the dose is 30 mg, the dose is 70 mg, the dose is 200 mg, or the dose is 600 mg.

In some embodiments of the present invention, the dose is administered once every three weeks. Further, in some embodiments, the dose is administered once every three weeks and the dose is in the range of 30 mg to 1800 mg, the dose is 900 mg, the dose is 1000 mg, the dose is 1200 mg, or the dose is 1800 mg.

In some embodiments of the present invention, the anti-TIM-3 antibody is administered with a loading dose followed by a maintenance dose, wherein the loading dose is administered once every two to three weeks for one to three cycles, the maintenance dose is administered once every two to three weeks following completion of the loading dose, the loading dose and the maintenance doses are in the range of 1 mg to 1800 mg, the maintenance dose is a lower dosage amount than the loading dose, and wherein the anti-human TIM-3 antibody comprises:
A.) a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7,
B.) a light chain variable region having the amino acid sequence of SEQ ID NO: 8 and a heavy chain variable region having the amino acid sequence of SEQ ID NO: 9, or
C.) a light chain having the amino acid sequence of SEQ ID NO: 10 and a heavy chain having the amino acid sequence of SEQ ID NO: 11.

Further, in some embodiments, the maintenance dose is half the dosage amount of the loading dose. In some embodiments, the loading dose is 1200 mg administered once every two weeks for two cycles, and the maintenance dose is 600 mg administered once every two weeks. In some embodiments, the loading dose is 1800 mg administered once every three weeks for three cycles, and the maintenance dose is 900 mg administered once every three weeks. Additionally, in some embodiments, the the maintenance dose is administered up to two years.

The present invention provides anti-human TIM-3 antibody for use in the treatment of cancer, wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody.

Further, in some embodiments, the anti-human PD-L1 antibody is durvalumab, atezolizumab, or avelumab. In some embodiments, the anti-human PD-L1 antibody comprises a light chain having the amino acid sequence of SEQ ID NO: 12 and a heavy chain having the amino acid sequence of SEQ ID NO: 13. In some embodiments, the anti-human PD-L1 antibody is administered at a dose of about 35 mg to about 1800 mg. Further, in some embodiments, the dose of the anti-human PD-L1 antibody is administered once every two weeks, and the dose is in the range of 70 mg to 700 mg, the dose is 35 mg, the dose is 70 mg, the dose is 200 mg, or the dose is 700 mg. In some embodiments, the dose of the anti-human PD-L1 antibody is administered once every three weeks, and the dose is in the range of 70 mg to 1400 mg, the dose is 1000 mg, the dose is 1200 mg, or the dose is 1400 mg. In some embodiments, the dose of the anti-human PD-L1 antibody is administered once every four weeks, and the dose is 1800 mg.

Further, in some embodiments, the anti-human PD-1 antibody administered is pembrolizumab, nivolumab, or cemiplimab. In some embodiments, the dose of the anti-human PD-1 antibody is 200 mg administered once every 3 weeks. In some embodiments, the dose of the anti-human PD-1 antibody is 240 mg administered once every 2 weeks or 480 mg once every 4 weeks.

The present invention provides an anti-human TIM-3 (SEQ ID NO: 1) antibody for use in the treatment of cancer, wherein the anti-human TIM-3 antibody is administered at a dose in the range of 1 mg to 1800 mg, and the anti-human TIM-3 antibody comprises a light chain having the amino acid sequence of SEQ ID NO: 10 and a heavy chain having the amino acid sequence of SEQ ID NO: 11, and wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, liver cancer, gallbladder cancer, cervical cancer, duodenal cancer, bone cancer, neuroendocrine cancer, intestinal cancer. Further, an embodiment of the present invention provides that the cancer melanoma or non-small cell lung cancer. Further, an embodiment of the present invention provides at least one of the anti-human TIM-3 antibody, anti-human PD-1 antibody, and anti-human PD-L1 antibody is administered with ionizing radiation.

The present invention provides an anti-human TIM-3 antibody for use in the treatment of cancer, wherein the anti-human TIM-3 antibody comprises a light chain having the amino acid sequence of SEQ ID NO: 10 and a heavy chain having the amino acid sequence of SEQ ID NO: 11, the anti-TIM-3 antibody is administered with a loading dose followed by a maintenance dose, wherein the loading dose is administered once every two to three weeks for one to three cycles, the maintenance dose is administered once every two to three weeks following completion of the loading dose, the loading dose and the maintenance doses are in the range of 1 mg to 1800 mg, the maintenance dose is a lower dosage amount than the loading dose, and wherein the the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, liver cancer, gallbladder cancer, cervical cancer, duodenal cancer, bone cancer, neuroendocrine cancer, intestinal cancer. Further, an embodiment of the present invention provides that the cancer melanoma or non-small cell lung cancer.

The present invention provides an embodiment wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody.

The present disclosure provides an anti-human TIM-3 (SEQ ID NO: 1) antibody for use in the treatment of a human patient whose cancer comprises a solid tumor that is mismatch repair deficient or exhibits a high degree of microsatellite instability; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody.

The present disclosure provides an anti-human TIM-3 (SEQ ID NO: 1) antibody for use in the treatment of a human patient whose cancer comprises a solid tumor; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7.

The present disclosure provides an anti-human TIM-3 (SEQ ID NO: 1) antibody for use in the treatment of a human patient whose cancer comprises a solid tumor; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a light chain variable region having the amino acid sequence of SEQ ID NO: 8 and a heavy chain variable region having the amino acid sequence of SEQ ID NO: 9.

The present disclosure provides an anti-human TIM-3 (SEQ ID NO: 1) antibody for use in the treatment of a human patient whose cancer comprises a solid tumor, an effective amount of an anti-human TIM-3 (SEQ ID NO: 1) antibody; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a light chain having the amino acid sequence of SEQ ID NO: 10 and a heavy chain having the amino acid sequence of SEQ ID NO: 11.

The present invention provides the use of an anti-human TIM-3 antibody for the manufacture of a medicament for the treatment of cancer comprising administering the anti-human TIM-3 antibody to a human patient in need thereof with a dose in the range of 1 mg to 1800 mg, and wherein the anti-human TIM-3 antibody comprises a light chain having the amino acid sequence of SEQ ID NO: 10 and a heavy chain having the amino acid sequence of SEQ ID NO: 11.

In some embodiments, the dose is administered once every two weeks. Further, in some embodiments, the dose is in the range of 30 mg to 1200 mg, the dose is 30 mg, the dose is 70 mg, the dose is 200 mg, or the dose is 600 mg.

In some embodiments, the dose is administered once every three weeks. Further, in some embodiments, the dose is in the range of 30 mg to 1800 mg, the dose is 900 mg, the dose is 1000 mg, the dose is 1200 mg, or the dose is 1800 mg.

The present invention provides the use of an anti-human TIM-3 antibody for the manufacture of a medicament for the treatment of cancer, wherein the anti-TIM-3 antibody is administered with a loading dose followed by a maintenance dose, wherein the loading dose is administered once every two to three weeks for one to three cycles, the maintenance dose is administered once every two to three weeks following completion of the loading dose, the loading dose and the maintenance doses are in the range of 1 mg to 1800 mg, the maintenance dose is a lower dosage amount than the loading dose, and wherein the anti-human TIM-3 antibody comprises:
A.) a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7,
B.) a light chain variable region having the amino acid sequence of SEQ ID NO: 8 and a heavy chain variable region having the amino acid sequence of SEQ ID NO: 9, or
C.) a light chain having the amino acid sequence of SEQ ID NO: 10 and a heavy chain having the amino acid sequence of SEQ ID NO: 11.

Further, in some embodiments, the maintenance dose is half the dosage amount of the loading dose. In some embodiments, the loading dose is 1200 mg administered once every two weeks for two cycles, and the maintenance dose is 600 mg administered once every two weeks. In some embodiments, the loading dose is 1800 mg administered once every three weeks for three cycles, and the maintenance dose is 900 mg administered once every three weeks. Additionally, in some embodiments, the the maintenance dose is administered up to two years.

The present disclosure provides the use of an anti-human TIM-3 antibody for the manufacture of a medicament for the treatment of cancer; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody.

The present disclosure provides the use of an anti-human TIM-3 antibody for the manufacture of a medicament for the treatment of a human patient; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7.

The present disclosure provides the use of an anti-human TIM-3 antibody for the manufacture of a medicament for the treatment of a human patient whose cancer comprises a solid tumor that is mismatch repair deficient or exhibits a high degree of microsatellite instability; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a light chain variable region having the amino acid sequence of SEQ ID NO: 8 and a heavy chain variable region having the amino acid sequence of SEQ ID NO: 9.

The present disclosure provides the use of an anti-human TIM-3 antibody for the manufacture of a medicament for the treatment of a human patient whose cancer comprises a solid tumor that is mismatch repair deficient or exhibits a high degree of microsatellite instability, an effective amount of an anti-human TIM-3 (SEQ ID NO: 1) antibody; wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody; wherein the anti-human TIM-3 antibody comprises a light chain having the amino acid sequence of SEQ ID NO: 10 and a heavy chain having the amino acid sequence of SEQ ID NO: 11.

The present provides a pharmaceutical composition comprising an anti-human TIM-3 antibody for use in the treatment of cancer in a human patient, wherein the anti-human TIM-3 antibody comprises a light chain having the amino acid sequence of SEQ ID NO: 10 and a heavy chain having the amino acid sequence of SEQ ID NO: 11, and wherein the anti-human TIM-3 antibody is administered at a dose of about 1 mg to about 1800 mg.

In some embodiments, the dose is administered once every two weeks. Further, in some embodiments, the dose is in the range of 30 mg to 1200 mg, the dose is 30 mg, the dose is 70 mg, the dose is 200 mg, or the dose is 600 mg.

In some embodiments, the dose is administered once every three weeks. Further, in some embodiments, the dose is in the range of 30 mg to 1800 mg, the dose is 900 mg, the dose is 1000 mg, the dose is 1200 mg, or the dose is 1800 mg.

The present provides a pharmaceutical composition comprising an anti-human TIM-3 antibody for use in the treatment of cancer in a human patient, wherein the anti-TIM-3 antibody is administered with a loading dose followed by a maintenance dose, wherein the loading dose is administered once every two to three weeks for one to three cycles, the maintenance dose is administered once every two to three weeks following completion of the loading dose, the loading dose and the maintenance doses are in the range of 1 mg to 1800 mg, and the maintenance dose is a lower dosage amount than the loading dose, and wherein the anti-human TIM-3 antibody comprises:
A.) a HCDR1 having the amino acid sequence of SEQ ID: 2, a HCDR2 having the amino acid sequence of SEQ ID NO: 3, a HCDR3 having the amino acid sequence of SEQ ID NO: 4, a LCDR1 having the amino acid sequence of SEQ ID NO: 5, a LCDR2 having the amino acid sequence of SEQ ID NO: 6, and a LCDR3 having the amino acid sequence of SEQ ID NO: 7,
B.) a light chain variable region having the amino acid sequence of SEQ ID NO: 8 and a heavy chain variable region having the amino acid sequence of SEQ ID NO: 9, or
C.) a light chain having the amino acid sequence of SEQ ID NO: 10 and a heavy chain having the amino acid sequence of SEQ ID NO: 11.

Further, in some embodiments, the maintenance dose is half the dosage amount of the loading dose. In some embodiments, the loading dose is 1200 mg administered once every two weeks for two cycles, and the maintenance dose is 600 mg administered once every two weeks. In some embodiments, the loading dose is 1800 mg administered once every three weeks for three cycles, and the maintenance dose is 900 mg administered once every three weeks. Additionally, in some embodiments, the the maintenance dose is administered up to two years.

The present disclosure further provides a pharmaceutical composition wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody. Further, an embodiment of the present invention provides a pharmaceutical composition wherein the anti-human PD-L1 antibody is administered at a dose of about 35 mg to about 1800 mg, about 70 mg to about 1400 mg, about 35 mg once every two weeks, about 70 mg once every three weeks, about 200 mg once every two weeks, 700 mg once every two weeks, about 1000 mg to about 100 mg, about 1000 mg once every three weeks, about 1400 mg once every three weeks, or about 1800 mg once every three weeks.

Further, an embodiment of the present invention provides a pharmaceutical composition, wherein the anti-human PD-L1 antibody is administered at a dose of 35 mg to 1800 mg, 70 mg to 1400 mg, 35 mg once every two weeks, 70 mg once every three weeks, 200 mg once every two weeks, 700 mg once every two weeks, 1000 mg to 100 mg, 1000 mg once every three weeks, 1400 mg once every three weeks, or 1800 mg once every three weeks, and wherein the anti-human PD-L1 antibody comprises a light chain having the amino acid sequence of SEQ ID NO: 12 and a heavy chain having the amino acid sequence of SEQ ID NO: 13.

Further, an embodiment of the present invention provides a pharmaceutical composition, wherein the anti-human PD-1 antibody is pembrolizumab, nivolumab, or cemiplimab. Further, an embodiment of the present invention provides a pharmaceutical composition, wherein the anti-human PD-1 antibody is pembrolizumab, nivolumab, or cemiplimab, and the dose of the anti-human PD-1 antibody is 200 mg administered once every 3 weeks, 240 mg administered once every 2 weeks, or 480 mg once every 4 weeks.

As used herein, the term “human TIM-3” refers to T-cell immunoglobulin and mucin-domain containing-3, having the amino acid sequence of SEQ ID NO: 1.

As used herein, the term “human PD-L1” refers to Human Programmed Death Receptor Ligand One, having the amino acid sequence of SEQ ID NO: 14.

As used herein, the term “human PD-1” refers to Human Programmed Death Receptor One, having the amino acid sequence of SEQ ID NO:15.

The anti-human antibodies disclosed herein are prefererably IgG-like antibodies and have “heavy” chains and “light” chains that are cross-linked via intra- and inter-chain disulfide bonds. Each heavy chain is comprised of an N-terminal heavy chain variable region (“HCVR”) and a heavy chain constant region (“HCCR”). Each light chain is comprised of a N-terminal light chain variable region (“LCVR”) and a light chain constant region (“LCCR”). When expressed in certain biological systems, antibodies having native human Fc sequences are glycosylated in the Fc region. Typically, glycosylation occurs in the Fc region of the antibody at a highly conserved N-glycosylation sites.

The HCVR and LCVR regions can be further subdivided into regions of hyper-variability, termed complementarity determining regions (“CDRs”), interspersed with regions that are more conserved, termed framework regions (“FR”). Each HCVR and LCVR is composed of three CDRs and four FRs, arranged from amino-terminus to carboxy-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues which form specific interactions with the antigen. For the purposes of anti-human TIM-3 antibody described and claimed via the CDRs herein, the North CDR definitions are used. The North CDR definition (North et al., “A New Clustering of Antibody CDR Loop Conformations”, Journal of Molecular Biology, 406, 228-256 (2011)) is based on affinity propagation clustering with a large number of crystal structures.

An isolated DNA encoding a HCVR region can be converted to a full-length heavy chain gene by operably linking the HCVR-encoding DNA to another DNA molecule encoding heavy chain constant regions. The sequences of human, as well as other mammalian, heavy chain constant region genes are known in the art. DNA fragments encompassing these regions can be obtained e.g., by standard PCR amplification.

An isolated DNA encoding a LCVR region may be converted to a full-length light chain gene by operably linking the LCVR-encoding DNA to another DNA molecule encoding a light chain constant region. The sequences of human, as well as other mammalian, light chain constant region genes are known in the art. DNA fragments encompassing these regions can be obtained by standard PCR amplification. The light chain constant region can be a kappa or lambda constant region. Preferably for the anti-human TIM-3 antibodies disclosed herein, the light chain constant region is a human kappa constant region.

As used herein, the terms “treating,” “treat,” or “treatment” refer to restraining, slowing, lessening, reducing, or reversing the progression or severity of an existing symptom, disorder, condition, or disease, or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In some examples, the present invention can be used as a medicament.

As used herein, the term “effective amount” refers to the amount or dose of an anti-human TIM-3 antibody, anti-human PD-L1 antibody, or anti-human PD-1 antibody, which provides an effective response in the patient under diagnosis or treatment.

As used herein, the term “effective response” of a patient or a patient's “responsiveness” to treatment with an agent and/or combination of agents refers to the clinical or therapeutic benefit imparted to a patient upon administration of an anti-human TIM-3 antibody and anti-human TIM-3 combinations with an anti-human PD-L1 antibody or an anti-human PD-1 antibody.

As used herein, the term “flat dose” or “flat dosing” or “fixed dose” or “fixed dosing” refers to dosing strategies without correction for body size or other (pharmacological) parameters.

As used herein, the term “loading dose” refers to the dose or doses designed to first be given to patients to rapidly achieve a certain desired level of drug before a maintenance regimen is then used for the patient. The loading dose or doses would be at a dosage higher than the maintenance dose, and would be given for a short duration of time, typically one to three cycles of administration.

As used herein, the term “maintenance dose” refers to the dose or doses designed to follow after the completion of the loading dose or doses for the patient. The maintenance dose is designed to sustain a desired drug level. The maintenance dose would be a lower dose amount or less frequent administration to the patient than the loading dose. The maintenance dose would be given for varied lengths of times or cycles of administration. The maintenance dose could be administered for one, two, three, four, five, ten, twenty, or more cycles, with the total time of the patient receiving the maintenance doses going to 3 months, 6 months, 9 months, 1 year, 2 years, or longer.

Generally, dosage regimens may be adjusted to provide the optimum desired response (e.g., a therapeutic response). Dosing amounts of the antibodies can also be adjusted by the physicians treating the patient and may fall within customary ranges.

The route of administration may be varied in any way, limited by the physical properties of the drugs and the convenience of the patient and the caregiver.

Preferably, the antibodies disclosed herein are formulated for intravenous administration. The antibodies may also be formulated for delivery via other parenteral routes such as subcutaneous delivery.

The therapeutically effective amount of the treatment of the invention can be measured by various endpoints commonly used in evaluating cancer treatments, including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, improved quality of life, and/or improving signs or symptoms of cancer.

As used herein, the term “progressive disease” (PD) refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term “complete response” (CR) refers to the disappearance of all target lesions with the short axes of any target lymph nodes reduced to <10 mm.

As used herein, the term “stable disease” (SD) refers to neither sufficient shrinkage to qualify for PR nor sufficient increase to qualify for PD, taking as reference the smallest sum of diameters while on study.

As used herein, the term “not evaluable” (NE) refers to when an incomplete radiologic assessment of target lesions is performed or there is a change in the method of measurement from baseline that impacts the ability to make a reliable evaluation of response.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. In a preferred embodiment, OS refers to the time from the date of randomization in the Study to the date of death from any cause. If the patient is alive at the end of the follow-up period or is lost to follow-up, OS data is censored on the last date the patient is known to be alive. Overall survival is evaluated by the Kaplan-Meier method, and a 95% confidence interval (CI) is provided for the median OS in each treatment arm.

As used herein, the term “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “disease control rate” (DCR) refers to lack of disease progression and rate thereof. It refers to the group of patients with a best overall response categorized as CR, PR or SD (specifically excluding the patients with PD), wherein the best overall response is the best response recorded from the start of treatment until PD.

As used herein, the term “clinical benefit rate,” refers to SD or better at 12 weeks. The tumor response rate of SD or better (i.e. CR+PR+SD) at 12 weeks is defined as the proportion of patients with a response of SD or better, as defined by RECIST 1.1, at 12 weeks following the first dose of study therapy. Patients will be considered “failure” if they die or if radiographic evaluation indicates a response of PD at 12 weeks or before.

As used herein, the term “extending survival” is meant as increasing OS or PFS in a treated patient relative to i) an untreated patient, ii) a patient treated with less than all of the anti-tumor agents in a particular combination therapy, or iii) a control treatment protocol. Survival is monitored following the initiation of treatment or following the initial diagnosis of cancer.

As used herein, the term “best overall response” is the best response recorded from the start of the study treatment until the earliest of objective progression or start of new anticancer therapy, taking into account any requirement for confirmation. The patient's best overall response assignment will depend on the findings of both target and nontarget disease and will also take into consideration the appearance of new lesions. The best overall response will be calculated via an algorithm using the assessment responses provided by the investigator over the course of the trial.

As used herein, the term “time-to-response” (TTR) is the time from the date of first study treatment until the first evidence of a confirmed CR or PR,

As used herein, the term “duration of response” (DoR) is defined only for responders (patients with a confirmed CR or PR). It is measured from the date of first evidence of a confirmed CR or PR to the date of the first observed radiographically documented PD, or the date of death due to any cause, whichever is earlier.

Microsatellites are short, tandem repeated DNA sequences that can be used to detect a form of genomic instability called microsatellite instability. The five mononucleotide repeat markers BAT-25 (GenBank® number L04143), BAT-26 (GenBank® number U41210), NR-21 (GenBank® number XM_033393), NR-24 (GenBank® number X60152), and MONO-27 (GenBank® number AC007684) are used herein to determine the status of the microsatellites in tumor samples (Bacher, Jeffery W. et al. “Development of a Fluorescent Multiplex Assay for Detection of MSI-High Tumors.” Disease markers 20.4-5 (2004): 237-250. PMC. Web. 2 Jan. 2018). The two pentanucleotide repeat markers Penta C (GenBank® number AL138752) and Penta D (GenBank® number AC0000114) are used to detect potential sample mixups or contamination. Microsatellite instability (MSI) status can be determined as performed herein using the commercially available test, the Promega MSI Analysis System, as per the manufacturer's directions (Promega Corp., Madison, Wis., catalog number MD1641).

“A high degree of microsatellite instability” (“MSI-H”) as used herein refers to a tumor sample having two or more of the mononucleotide repeat markers: BAT-25 (GenBank® number L04143), BAT-26 (GenBank® number U41210), NR-21 (GenBank® number XM_033393), NR-24 (GenBank® number X60152), and MONO-27 (GenBank® number AC007684). “Microsatellite Stable” (“MSS”) as used herein refers to a tumor sample in which none of the aforementioned markers are altered.

“Mismatch Repair Deficient” (“MMR-D”) as used herein refers to a tumor sample that contains a mutation in or is deficient in the repair of of one or more of the following DNA replication genes: MLH1, MSH2, MSH6, and PMS2. The presence of altered or mutated versions of these proteins in the tumor can be detected by immunohistochemistry (“IHC”) assays. In some examples, the patient whose tumor is MSI-high or MMR-D may also have a tumor microenvironment that expresses high levels of PD-L1. PD-L1 expression on tumors and infiltrating immune cells is assessed herein using the SP-263 PD-L1 immunohistochemical assay (Roche Catalog Number 740-4907), per manufacturer's instructions. As the assay has not yet been validated for all solid tumors, an exploratory cutoff that utilized the results from both immune and tumor cell staining is employed: as used herein, “PD-L1 High” refers to a percentage of 25% or greater tumor cells with any membrane staining for PD-L1 above background or a percentage of 25% or greater of tumor-associated immune cells with any positive staining for PD-L1 at any intensity above background. As used herein, “PD-L1 Low” refers to a percentage of less than 25% of tumor cells with any membrane staining for PD-L1 above background or a percentage of less than 25% of tumor-associated immune cells with any positive staining for PD-L1 at any intensity above background.

The antibodies described herein may readily be produced in mammalian cells, non-limiting examples of which includes CHO, NS0, HEK293 or COS cells. The host cells are cultured using techniques well known in the art. In this regard, an appropriate host cell can be either transiently or stably transfected with an expression system for secreting antibodies using an optimal predetermined HC:LC vector ratio or a single vector system encoding both HC (heavy chain) and LC (light chain). The vectors containing the polynucleotide sequences of interest (e.g., the polynucleotides encoding the polypeptides of the antibody and expression control sequences) can be transferred into the host cell by well-known methods, which may vary depending on the type of cellular host. Clarified media, into which the antibody has been secreted, may be purified using any of many commonly-used techniques. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994). In some examples, the medium may be conveniently applied to a column that has been equilibrated with a compatible buffer. The column may be washed to remove nonspecific binding components. The bound antibody may be eluted, for example, by pH gradient. Antibody fractions may be detected, such as by UV absorbance or SDS-PAGE, and then may be pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the antibody after these chromatography steps is typically greater than 95%. The product may be immediately frozen at −70° C. or may be lyophilized.

In embodiments that refer to a method of treatment as described herein, such embodiments are also further embodiments for use in that treatment, or alternatively for the use of the combination for the manufacture of a medicament for use in that treatment.

A Phase 1a/1b Study of an Anti-Human TIM-3 Antibody (LY3321367), Administered Alone or in Combination with LY3300054, an Anti-Human PD-L1 Antibody, in Advanced Relapsed/Refractory Solid Tumors (NCT03099109).

The study is a multicenter, nonrandomized, open-label Phase 1a/1b study in patients with advanced solid tumors. The Phase 1a dose escalation will assess the safety and tolerability of an anti-human TIM-3 antibody having the light chain amino acid sequence of SEQ ID: 10 and the heavy chain amino acid sequence of SEQ ID: 11 administered as monotherapy (cohort A) and in combination with an anti-human PD-L1 antibody having the light chain amino acid sequence of SEQ ID: 12 and the heavy chain amino acid sequence of SEQ ID: 13 (cohort B), in patients with advanced relapsed/refractory solid tumors. The Phase 1b dose expansion will assess the safety and tolerability of an anti-human TIM-3 antibody administered as monotherapy (cohort A) and in combination with an anti-PD-L1 antibody in patients with advanced relapsed/refractory solid tumors (cohort B). Approximately 33 patients are enrolled in the Phase 1a, and approximately 130 patients are enrolled in the Phase 1b. Patients enrolled in Phase 1a (Treatment Arm A or Treatment Arm B) will receive anti-human TIM-3 monotherapy or in combination with an anti-human PD-L1 antibody, as shown in Table 1 below.

TABLE 1
Treatment Arms and Dosing Regimens for
the phase 1a/b Study NCT03099109
Treatment Arm A Cohorts:
Anti-human TIM-3 Monotherapy

| | Anti-TIM-3 Dose | Dosing |
|---|---|---|
| Cohort | (mg, intravenous (IV)) | Schedule |
| A-1 | 1 | D 1 (Day 1)and D 15 (Day |
| A1 | 3 (D 1 only) | 15) of each |
| | 10 (subsequent doses) | 28-day cycle |
| A2 | 30 | |
| A3 | 70 | |
| A4 | 200 | |
| A5 | 600 | |
| A6 | 1800 | D 1 of each 21-day |
| | | cycle for 3 cycles |
| | | followed by 900 mg |
| | | D 1 of each 28-day cycle |
| A7 | 1200 | D 1 and D 15 of each |
| | | 28-day cycle for 2 |
| | | cycles followed by |
| | | 600 mg D 1 and D 15 of |
| | | each 28-day cycle |

Treatment Arm B Cohorts: anti-human TIM-3 antibody
in Combination with anti-human PD-L1 antibody

| | Anti-TIM-3 Dose | Anti-PD-L1 Dose | |
|---|---|---|---|
| Cohort | (mg, IV) | (mg, IV) | Dosing |
| B1 | 30 | 200 | D 1 and D 15 |
| B2 | 70 | 200 | of each 28- |
| B3 | 70 | 700 | day cycle |
| B4 | 200 | 700 | |
| B5 | 600 | 700 | |
| B6 | 1800 mg D 1 of each 21-day | 1400 | D 1 of each |
| | cycle for 3 cycles followed | | 21-day |
| | by 900 mg D 1 of each 21-day | | cycleb |
| | cycle | | |
| B7 | 1200 mg D 1 and Day 15 of | 700 | D 1 and D 15 |
| | each 28-day cycle for 2 | | of each 28- |
| | cycles followed by 600 mg | | day cycle |
| | D 1 and D 15 of each 28-day | | |
| | cycle | | |

Q2W means administered every 2 weeks. Q3W means administered every 3 weeks. Q4W means administered every 4 weeks. MTD means the maximum tolerated dose.

For the clinical trial, the anti-human TIM-3 antibody has been dosed up to 1200 mg Q2W by IV infusion for cycles 1 and 2, followed by 600 mg Q2W by IV infusion for cycle 3 onward as monotherapy, and in combination with anti-human PD-L1 antibody (700 mg Q2W). LY3321367 t½ was ˜22 days. LY3321367 PK did not vary when it was administered in combination with anti-human PD-L1 (LY3300054). Based on PK/pharmacodynamic modeling results, it is predicted that the anti-human TIM-3 antibody at 1200 mg Q2W for cycles 1 and 2 followed by 600 mg Q2W for cycle 3 onward neutralizes >99% of human TIM-3, both in the blood stream and on the cell surface.

A Phase 1a/1b Study of a Anti-PD-L1 Checkpoint Antibody Administered Alone or in Combination with Other Agents in Advanced Refractory Solid Tumors (Phase 1a/1b Anti-PD-L1 Combinations in Tumors-PACT) (NCT02791334).

The Study is a multicenter 2 part Phase 1a dose escalation and a Phase 1b dose expansion study. The Phase 1b dose expansion study assesses the safety, tolerability, and efficacy of an anti-human PD-L1 antibody having the light chain amino acid sequence of SEQ ID: 12 and the heavy chain amino acid sequence of SEQ ID: 13 as monotherapy in melanoma and MSI-H solid tumors, and in combination with an anti-human TIM-3 antibody having the light chain amino acid sequence of SEQ ID: 10 and the heavy chain amino acid sequence of SEQ ID: 11 in patients with histologically or cytologically confirmed diagnosis of advanced solid tumor shown to be MSI-H or MMR-D. Two combination expansions will be investigated. The first combination expansion includes MSI-H or MMR-D cancer patients previously untreated with an anti-human PD-1 or an anti-human PD-L1 antibody (PD-1/PD-L1 naïve), and the second combination expansion includes MSI-H or MMR-D cancer patients that are resistant and/or refractory to treatment with anti-human PD-1 and/or anti-human PD-L1 antibodies. Approximately 20 patients are enrolled in the expansion cohorts with the potential to enroll 20 additional patients depending on results of a data review (including but not limited to safety, efficacy, and pharmacokinetics/pharmacodynamics).

The Phase 1a monotherapy patients receive an anti-human PD-L1 antibody at 70 mg, 200 mg, or 700 mg Q2W; or 1000 mg or 1400 mg every 21 days (Q3W) by IV infusion; or 1800 mg every 28 days (Q4W) by IV infusion.

The Phase 1a monotherapy patients receive the anti-human PD-L1 antibody at a starting dose of 70 mg (equivalent to 1 mg/kg for a 70-kg human) Q2W by IV infusion. If the dose is well-tolerated, a stepwise dose escalation is conducted at 200 mg and 700 mg (equivalent to 3 mg/kg and 10 mg/kg for a 70-kg human, respectively), administered Q2W by IV infusion.

Preliminary data suggests the anti-human PD-L1 antibody is well tolerated up at 700 mg Q2W and 1000 mg Q3W as a monotherapy. Using the preliminary population PK model for the anti-human PD-L1 antibody, the regimen of 1400 mg Q3W is expected to have a steady-state trough concentration similar to that of the 700 mg Q2W, and therefore is viewed as a conservative surrogate regimen for 700 mg Q2W as monotherapy or in combination.

Using the preliminary population PK model for the anti-human PD-L1 antibody regiment, the regimen of 1800 mg Q4W is expected to have a steady-state trough concentration approximately 75% of that from 700 mg Q2W, and therefore is viewed as a possible surrogate regimen for 700 mg Q2W. Based on safety, tolerability, and efficacy of the dosing regimen of 700 mg Q2W, the anti-human PD-L1 antibody is the recommended phase 2 dose.

TABLE 2
Model-Predicted anti-human PD-L1 antibody Pharmacokinetic
Descriptors Post the First Dose and at Steady State

| | Post-First Dose^{a} | | | Steady State^{a} | | | |
|---|---|---|---|---|---|---|---|
| | Cₘₐₓ | C_{trough} | AUC_{τ} | Cₘₐₓ | C_{trough} | AUCτ | |
| | (μg/mL) | (μg/mL) | (μg · day/mL) | (μg/mL) | (μg/mL) | (μg · day/mL) | R^{b} |
| 70 | 22.6 | 5.41 | 135 | 39.6 | 15.8 | 318 | 2.36 |
| mg | (11.6-42.4) | (2.62-9.58) | (93.3-206) | (25.3-64.4) | (6.60-33.2) | (184-572) | |
| Q2W | | | | | | | |
| 200 | 65.0 | 15.6 | 390 | 112 | 45.2 | 902 | 2.31 |
| mg | (35.0-123) | (6.91-27.3) | (264-574) | (72.0-182) | (16.3-93.4) | (479-1620) | |
| Q2W | | | | | | | |
| 700 | 231 | 55.4 | 1380 | 405 | 163 | 3300 | 2.39 |
| mg | (123-444) | (25.8-95.3) | (938-2000) | (253-645) | (61.5-327) | (1710-5670) | |
| Q2W | | | | | | | |
| 1000 | 319 | 60.8 | 2420 | 478 | 138 | 4660 | 1.93 |
| mg | (167-608) | (25.7-116) | (1640-3880) | (301-762) | (44.0-302) | (2470-8380) | |
| Q3W | | | | | | | |
| 1400 | 444 | 83.7 | 3410 | 653 | 184 | 6390 | 1.87 |
| mg | (242-816) | (37.7-154) | (2240-5260) | (415-1070) | (66.5-415) | (3530-11600) | |
| Q3W | | | | | | | |
| 1800 | 593 | 83.6 | 4940 | 764 | 160 | 8200 | 1.66 |
| mg | (314-1100) | (32.0-163) | (3160-7810) | (465-1280) | (42.3-366) | (4390-14700) | |
| Q4W | | | | | | | |

Abbreviations: AUCτ=area under the concentration-time curve in a dosing interval (336 hr for Q2W dosing, 504 hr for Q3W dosing, and 672 hr for Q4W);
Cₘₐₓ=maximum drug concentration (assumed to be at end of infusion); C_{trough}=trough concentration; Q2W=every 2 weeks; Q3W=every 3 weeks; Q4W=every 4 weeks; R=accumulation ratio. ^{a}Shown as median (5% and 95% quantiles) summarized from 1000 simulation repetitions for each regimen. ^{b}Accumulation ratio: calculated as AUCτ (steady state)/AUCτ (post first dose).

Based on PK/pharmacodynamic modeling exercises conducted in a data review of an ongoing Phase 1 Study NCT03099109, it is predicted that the anti-human TIM-3 antibody at 1200 mg Q2W for cycles 1 and 2 followed by 600 mg Q2W for cycle 3 onward can neutralize >99% of TIM-3 both in blood stream and on cell surface.

Patients in the Phase 1b dose expansion combination receive 700 mg anti-human PD-L1 antibody Q2W and 1200 mg of the anti-human TIM-3 antibody for cycles 1 and 2 Q2W followed by 600 mg of the anti-human TIM-3 antibody for cycle 3 onward, given Q2W via IV infusion.

TABLE 4
Phase 1a Treatment Arms and Dosing Regimens

| Phase 1a- | Study Drug(s) | | Corresponding | | | Treatment |
|---|---|---|---|---|---|---|
| Treatment | (route of | | Dose Levels | Dose | Dose | Cycle |
| Arm | administration) | Cohorts | (mg) | Frequency | Day | Duration |
| A | Anti-PD-L1 | A(-1)^{a}, | 35, 70, 200, | Q2W | D 1, | 28 Days |
| | Antibody (IV) | A1, A2, | 700 | | D 15 | |
| | | A3 | | | | |
| A-Q3W | Anti-PD-L1 | A-Q3W | 1000^{b} | Q3W | D 1 | 21 Days |
| | Antibody (IV) | | | | | |
| A-Q4W | Anti-PD-L1 | A-Q4W | 1800^{c} | Q4W | D 1 | 28 Days |
| | Antibody (IV) | | | | | |

TABLE 5
Dose Expansion Phase 1b Treatment Arms and Dosing Regimens

| Phase 1b | Study Drug(s) | | Dose | | | Treatment |
|---|---|---|---|---|---|---|
| Treatment | (Route of | | Level | Dose | Dose | Cycle |
| Arm | Administration) | Cohorts | (mg) | Frequency | Day | Duration |
| A | Anti-human | A4 (melanoma), | 700 | Q2W | D 1, 15 | 28 |
| | PD-L1 | A5 (MSI-H) | | | | Days |
| | Antibody (IV) | | | | | |
| E | Anti-human | E1 (PD-1/PD-L1- | 1200 | Q2W | D 1, 15 | 28 |
| | TIM-3 | naïve, MSI-H), | Q2W | | | Days |
| | Antibody (IV) | E2 (PD-1/PD-L1- | Cycles 1-2; | | | |
| | | resistant/refractory, | 600 | | | |
| | | MSI-H) | Q2W | | | |
| | | | Cycles ≥3 | | | |
| | Anti-human | | 700 | Q2W | D 1, 15 | 28 |
| | PD-L1 | | | | | Days |
| | Antibody (IV) | | | | | |

For the primary objective, patients will be followed for at least 24 weeks of treatment after the last patient is enrolled. For the secondary objective of progression-free survival (PFS) and tertiary objectives of overall survival (OS), patients will be followed for their status until death or until study completion, whichever occurs first. The Phase 1b data will provide additional safety, tolerability, PK, pharmacodynamics, and efficacy data for the monotherapy and combinations.

The treatment period begins with the day of the patient's first study treatment and ends the day the patient and investigator agree that the patient will discontinue study treatment (discontinuation of assigned study drug(s)). Individual patients who enroll in this study may continue treatment until they have confirmed progressive disease, completed 12 months of study treatment (longer duration may be considered for a patient receiving clinical benefit, in consultation with the Lilly CRP), or discontinued study treatment for any other reason.

The end of trial is defined as the last patient's last visit. The End of Trial occurs after study completion, and after the last patient has discontinued study treatment and completed any applicable continued access follow-up visits.

### Microsatellite Instability Detection

Microsatellite instability can be assessed as follows. Briefly, the Promega MSI Analysis System, Version 1.2, is a fluorescent PCR-based assay for detection of microsatellite instability. Typically, MSI analysis involves comparing allelic profiles of microsatellite markers generated by amplification of DNA from matching normal and test samples, which may be MMR deficient. Alleles that are present in the test sample but not found in the corresponding normal samples indicate MSI. The MSI Analysis System includes fluorescently labeled primers for co-amplification of seven markers including five mononucleotide repeat markers (BAT-25, BAT-26, NR-21, NR-24 and MONO-27) and two pentanucleotide repeat markers (Penta C and Penta D). The mononucleotide markers are used for MSI determination, and the pentanucleotide markers are used to detect potential sample mixups and/or contamination. Internal lane size standards are added to the PCR samples to assure accurate sizing of alleles and to adjust for run-to-run variation. The PCR products are separated by capillary electrophoresis using an ABI PRISM® 310, 3100 or 3100-Avant Genetic Analyzer or Applied Biosystems 3130 or 3130x/Genetic Analyzer or equivalent. The output data may be analyzed with GeneMapper® Analysis Software to determine MSI status.

### Mismatch Repair Deficient Detection

The state of mismatch repair deficiency can be assessed as follows. Immunohistochemistry staining is used for the detection of mismatch repair deficiency in the tumor samples. Briefly, tumor samples are formalin-fixed, paraffin-embedded, or are placed on slides for analysis. The slides with the tumor samples are air-dried and not oven-baked. The samples are then stained for the mismatch repair protein markers MLH1, MSH2, MSH6, and PMS2 to determine the presence or absence of the mismatch repair proteins.

### PD-L1 and PD-1 Expression Analysis

PD-L1 expression can be determined as follows. PD-L1 expression on tumors and infiltrating immune cells is assessed using the SP-263 PD-L1 immunohistochemical assay (Roche Catalog Number 740-4907). As the assay has not yet been validated for all solid tumors, an exploratory cutoff that utilized the results from both immune and tumor cell staining is employed: “PD-L1 High” refers to a percentage of 25% or greater tumor cells with any membrane staining for PD-L1 above background or a percentage of 25% or greater of tumor-associated immune cells with any positive staining for PD-L1 at any intensity above background. “PD-L1 Low” refers to a percentage of less than 25% of tumor cells with any membrane staining for PD-L1 above background or a percentage of less than 25% of tumor-associated immune cells with any positive staining for PD-L1 at any intensity above background. PD-L1 expression on tumors and infiltrating immune cells can also be assessed using the 22C3 test, a qualitative immunohistochemical assay using Monoclonal Mouse Anti-PD-L1, Clone 22C3 intended for use in the detection of PD-L1 protein in formalin-fixed, paraffin-embedded developed by Dako (PD-L1 IHC 22C3 pharmDx, Agilent Dako) as per manufacturer's instructions. As the assay has not yet been validated for all solid tumors, PD-L1 expression is determined by using a Tumor Proportion Score (TPS), which is the percentage of viable tumor cells showing partial or complete membrane staining at any intensity PD-L1 positive if TPS ≥1%.

SEQUENCE LISTING

SEQ ID NO: 1 (Human TIM-3 extracellular domain
including signal peptide)
MFSHLPFDCVLLLLLLLLTRSSEVEYRAEVGQNAYLPCFYTPAAPGNLV
PVCWGKGACPVFECGNVVLRTDERDVNYWTSRYWLNGDFRKGDVSLTIE
NVTLADSGIYCCRIQIPGIMNDEKFNLKLVIK
SEQ ID NO: 2 (HCDR1 anti-Human TIM-3)
AASGFTFSSYYMS
SEQ ID NO: 3 (HCDR2 anti-Human TIM-3)
AISGSGGSTYYADSVKG
SEQ ID NO: 4 (HCDR3 anti-Human TIM-3)
ARYARTAFDL
SEQ ID NO: 5 (LCDR1 anti-Human TIM-3)
QASQDIYNYLN
SEQ ID NO: 6 (LCDR2 anti-Human TIM-3)
YAASSLQS
SEQ ID NO: 7 (LCDR3 anti-Human TIM-3)
QQANSFPPT
SEQ ID NO: 8 (Anti-Human TIM-3 Antibody, LCVR)
DIVMTQSPSSLSASVGDGVTITCQASQDIYNYLNWYQQKPGKAPKLLIY
AASSLQSGVPSRFSGSGSGTDFTLTISSLQPEDFATYYCQQANSFPPTF
GQGTKLEIK
SEQ ID NO: 9 (Anti-Human TIM-3 Antibody, HCVR)
EVQLLESGGGLVQPGGSLRLSCAASGFTFSSYYMSWVRQAPGKGLEWVS
AISGSGGSTYYADSVKGRETISRDNSKNTLYLQMNSLRAEDTAVYYCAR
YARTAFDLWGQGTLVTVSS
SEQ ID NO: 10 (Anti-Human TIM-3 Antibody, LC)
DIVMTQSPSSLSASVGDGVTITCQASQDIYNYLNWYQQKPGKAPKLLIY
AASSLQSGVPSRFSGSGSGTDFTLTISSLQPEDFATYYCQQANSEPPTE
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 11 (Anti-Human TIM-3 Antibody, HC)
EVQLLESGGGLVQPGGSLRLSCAASGFTFSSYYMSWVRQAPGKGLEWVS
AISGSGGSTYYADSVKGRETISRDNSKNTLYLQMNSLRAEDTAVYYCAR
YARTAFDLWGQGTLVTVSSASTKGPSVFPLAPSSKSTSGGTAALGCLVK
DYFPEPVTVSWNSGALTSGVHTFPAVLQSSGLYSLSSVVTVPSSSLGTQ
TYICNVNHKPSNTKVDKRVEPKSCDKTHTCPPCPAPEAEGAPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPSSIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGK
SEQ ID NO:12 (Anti-Human PD-L1 Antibody, LC)
(Artificial Sequence)
QSVLTQPPSASGTPGQRVTISCSGSSSNIGSNTVNWYQQLPGTAPKLLI
YGNSNRPSGVPDRFSGSKSGTSASLAISGLQSEDEADYYCQSYDSSLSG
SVFGGGIKLTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPAECS
SEQ ID NO: 13 (Anti-Human PD-L1 Antibody, HC)
(Artificial Sequence)
QVQLVQSGAEVKKPGSSVKVSCKASGGTFSSYAISWVRQAPGQGLEWMG
GIIPIFGTANYAQKFQGRVTITADKSTSTAYMELSSLRSEDTAVYYCAR
SPDYSPYYYYGMDVWGQGTTVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKRVEPKSCDKTHTCPPCPAPEAEGAPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPSSIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
SEQ ID NO: 14 (Human PD-L1) (Homo Sapiens)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET
SEQ ID NO: 15 (Human PD-1) (Homo Sapiens)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL

1. A method of treating cancer comprising administering an anti-human TIM-3 antibody to a human patient in need thereof with a dose in the range of 1 mg to 1800 mg, wherein the anti-human TIM-3 antibody comprises a light chain having the amino acid sequence of SEQ ID NO: 10 and a heavy chain having the amino acid sequence of SEQ ID NO: 11.

2. The method of claim 1, wherein the dose is administered once every two weeks.

3. The method of claim 2, wherein the dose is in the range of 30 mg to 1200 mg.

4. The method of claim 2, wherein the dose is 30 mg.

5. The method of claim 2, wherein the dose is 70 mg.

6. The method of claim 2, wherein the dose is 200 mg.

7. The method of claim 2, wherein the dose is 600 mg.

8. The method of claim 1, wherein the dose is administered once every three weeks.

9.-13. (canceled)

14. The method of claim 1, wherein the anti-TIM-3 antibody is administered with a loading dose followed by a maintenance dose, wherein the loading dose is administered once every two to three weeks for one to three cycles, the maintenance dose is administered once every two to three weeks following completion of the loading dose, the loading dose and the maintenance doses are in the range of 1 mg to 1800 mg, and the maintenance dose is a lower dosage amount than the loading dose.

15. (canceled)

16. The method of claim 14, wherein the loading dose is 1200 mg administered once every two weeks for two cycles, and the maintenance dose is 600 mg administered once every two weeks.

17. The method of claim 14, wherein the loading dose is 1800 mg administered once every three weeks for three cycles, and the maintenance dose is 900 mg administered once every three weeks.

18. (canceled)

19. The method of claim 1, wherein the anti-human TIM-3 antibody is administered in combination with an effective amount of an anti-human PD-L1 (SEQ ID NO: 14) antibody or an effective amount of an anti-human PD-1 (SEQ ID NO: 15) antibody.

20. The method of claim 19, wherein the anti-human PD-L1 antibody is durvalumab, atezolizumab, or avelumab.

21. The method of claim 19, wherein the anti-human PD-L1 antibody comprises a light chain having the amino acid sequence of SEQ ID NO: 12 and a heavy chain having the amino acid sequence of SEQ ID NO: 13.

22. The method of claim 19, wherein the anti-human PD-L1 antibody is administered at a dose of about 35 mg to about 1800 mg.

23. The method of claim 22, wherein the dose of the anti-human PD-L1 antibody is administered once every two weeks.

24.-35. (canceled)

36. The method of claim 19, wherein the anti-human PD-1 antibody is administered and is pembrolizumab, nivolumab, or cemiplimab.

37.-39. (canceled)

40. The method of claim 1, wherein the patient was previously treated with an anti-human PD-L1 antibody or an anti-human PD-1 antibody.

41. The method of claim 1, wherein the patient has not been previously treated with an anti-human PD-L1 antibody or an anti-human PD-1 antibody.

42. The method of claim 1, wherein the cancer is a solid tumor.

43. The method of claim 42, wherein the solid tumor is PD-L1 high.

44. The method of claim 42, wherein the solid tumor is PD-L1 low.

45. The method of claim 42, wherein the solid tumor has a high degree of microsatellite instability.

46. The method of claim 42, wherein the solid tumor is mismatch repair deficient.

47. The method of claim 42, wherein the solid tumor has a high degree of microsatellite instability and is mismatch repair deficient.

48. The method of claim 1, wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, liver cancer, gallbladder cancer, cervical cancer, duodenal cancer, bone cancer, neuroendocrine cancer, intestinal cancer.

49. The method of claim 48, wherein the cancer is melanoma or non-small cell lung cancer.

50. The method of claim 1, wherein the anti-human TIM-3 antibody is administered with ionizing radiation.

51.-150. (canceled)


SEQUENCE LISTING (15 sequences)

SEQ ID NO: 1 - protein, 130 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 2 - protein, 13 aa, Artificial Sequence
  SYNTHETIC
AASGFTFSSYYMS

SEQ ID NO: 3 - protein, 17 aa, Artificial Sequence
  SYNTHETIC
AISGSGGSTYYADSVKG

SEQ ID NO: 4 - protein, 10 aa, Artificial Sequence
  SYNTHETIC
ARYARTAFDL

SEQ ID NO: 5 - protein, 11 aa, Artificial Sequence
  SYNTHETIC
QASQDIYNYLN

SEQ ID NO: 6 - protein, 8 aa, Artificial Sequence
  SYNTHETIC
YAASSLQS

SEQ ID NO: 7 - protein, 9 aa, Artificial Sequence
  SYNTHETIC
QQANSFPPT

SEQ ID NO: 8 - protein, 107 aa, Artificial Sequence
  SYNTHETIC
DIVMTQSPSSLSASVGDGVTITCQASQDIYNYLNWYQQKPGKAPKLLIYAASSLQSGVPSRFSGSGSGTDFTLTISSLQPEDFATYYCQQANSFPPTFGQGTKLEIK

SEQ ID NO: 9 - protein, 117 aa, Artificial Sequence
  SYNTHETIC
EVQLLESGGGLVQPGGSLRLSCAASGFTFSSYYMSWVRQAPGKGLEWVSAISGSGGSTYYADSVKGRFTISRDNSKNTLYLQMNSLRAEDTAVYYCARYARTAFDLWGQGTLVTVSS

SEQ ID NO: 10 - protein, 214 aa, Artificial Sequence
  SYNTHETIC
DIVMTQSPSSLSASVGDGVTITCQASQDIYNYLNWYQQKPGKAPKLLIYAASSLQSGVPSRFSGSGSGTDFTLTISSLQPEDFATYYCQQANSFPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 447 aa, Artificial Sequence
  SYNTHETIC
EVQLLESGGGLVQPGGSLRLSCAASGFTFSSYYMSWVRQAPGKGLEWVSAISGSGGSTYYADSVKGRFTISRDNSKNTLYLQMNSLRAEDTAVYYCARYARTAFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Artificial Sequence
  SYNTHETIC
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 13 - protein, 453 aa, Artificial Sequence
  SYNTHETIC
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL